(19) United States
(12) Patent Application Publication
(10) Pub. No.: US 2023/0313104 A1
(43) Pub. Date: Oct. 5, 2023

(54) INTEGRATED 3D CELL CULTURE MATRIX AND EPITHELIAL SUPPORT LAYER

(71) Applicant: The Charles Stark Draper Laboratory, Inc., Cambridge, MA (US)

(72) Inventors: Rebecca CHRISTIANSON, Southborough, MA (US); Elizabeth WIELLETTE, Boston, MA (US); Melissa SPRACHMAN, Somerville, MA (US); Alicica M. MEEHAN-QIU, Somerville, MA (US); Lauren A. HAPACH, Boston, MA (US); Peter HSI, Waltham, MA (US); Hesham AZIZGOLSHANI, Cambridge, MA (US)

(21) Appl. No.: 18/129,344

(22) Filed: Mar. 31, 2023

Related U.S. Application Data
(60) Provisional application No. 63/326,741, filed on Apr. 1, 2022.

(51) Int. Cl. C12M 1/12; D01D 5/00; C12M 3/00

(52) U.S. Cl. CPC C12M 25/14; C12M 25/02; D01D 5/0007; C12M 21/08; D10B 2321/12; D10B 2331/041

(57) ABSTRACT

A method for generating a cell support interface for use in a three dimensional cell culture environment, may include electrospinning a mat having an epithelial support layer configured to create an intimate coupling between the epithelial cell and a porous matrix, including a first layer and a second layer, wherein the first layer is formed using a first solution at a first viscosity level and the second layer is formed using a second solution at a second viscosity level different from the first viscosity level.

### CROSS-REFERENCE TO RELATED APPLICATIONS

This application claims the benefit of U.S. provisional application Ser. No. 63/326,741 filed Apr. 1, 2022, the disclosure of which is hereby incorporated in its entirety by reference herein.

### TECHNICAL FIELD

Disclosed herein are systems and methods for integrated 3D cell culture matrix and epithelial support layers.

### BACKGROUND

In vitro tissue and organ-on-a-chip models are useful tools to model the behavior of biologic tissue under various conditions. It is challenging to model complex tissues using such techniques.

### SUMMARY

A method for generating a cell support interface for use in a three dimensional cell culture environment, may include electrospinning a mat having an epithelial support layer configured to create an intimate coupling between the epithelial cell and a porous matrix, including a first layer and a second layer, wherein the first layer is formed using a first solution at a first viscosity level and the second layer is formed using a second solution at a second viscosity level different from the first viscosity level.

A method for generating an electrospun mat for use in a three dimensional cell culture environment, may include electrospinning a mat to facilitate intimate coupling between epithelial cell and a porous matrix, including co-spinning a first solution and a second solution, wherein the mat includes an epithelial support layer configured to intimately mate with epithelial cells cultured thereon.

A method for generating a cell support interface for use in a three dimensional cell culture environment, may include electrospinning a first layer using a first solution at a first viscosity level; and electrospinning a second layer using a second solution at a second viscosity level different from the first viscosity level, where the first layer and the second layer result in differing porosity levels for the first and second layers.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing.

In the drawings:

FIG. 1 is a block diagram of an example interface that implements the techniques described herein, according to one or more implementations;

FIG. 2A illustrates an example drum collector in an electrospinning setup, according to one or more implementations;

FIG. 2B illustrates an example of electrospun Poly(ε-caprolactone) (PCL) fibers, according to one or more implementations;

FIG. 2C illustrates a non-magnified image of an example electrospun PCL mat, according to one or more implementations;

FIG. 3A illustrates an example of a thinner layer of material collected directly onto a porous material appropriate to support epithelial cells, such as track etched polycarbonate, according to one or more implementations;

FIG. 3B illustrates an example of a thinner layer of material collected directly onto a porous material appropriate to support epithelial cells, such as track etched polycarbonate according to one or more implementations;

FIG. 3C illustrates an example of a thicker layer of material collected directly on onto a porous material appropriate to support epithelial cells, such as track etched polycarbonate according to one or more implementations;

FIG. 3D illustrates an example of a thicker layer of material collected directly onto a porous material appropriate to support epithelial cells, such as track etched polycarbonate according to one or more implementations;

FIG. 3E illustrates an example of a thicker layer of material collected directly onto a porous material appropriate to support epithelial cells, such as track etched polycarbonate according to one or more implementations;

FIG. 3F illustrates an example of a thinner layer of material collected directly onto a porous material appropriate to support epithelial cells, such as track etched polycarbonate, in other implementations;

FIG. 4 illustrates a general stack-up arrangement for an example configuration resulting in electrospun mats depositing at differing porosities, according to one or more implementations;

FIG. 5A shows example scanning electron microscopy (SEM) images of PCL fibers generated from electrospinning PCL onto to the collector as configured in FIG. 4, demonstrating a lower porosity layer according to one or more implementations;

FIG. 5B shows example scanning electron microscopy (SEM) images of PCL fibers generated from electrospinning PCL onto to the collector as configured in FIG. 4, demonstrating a higher porosity layer according to one or more implementations;

FIG. 6 illustrates an example stack-up arrangement for an example configuration of the layers, according to one or more implementations;

FIG. 7A illustrates an example of an electrospun PCL mat electrospun directly onto a foil-wrapped collector using parameters according to one or more implementations;

FIG. 7B illustrates an example of an electrospun PCL mat electrospun directly onto a foil-wrapped collector using different parameters than FIG. 7A, according to one or more implementations;

FIG. 8A illustrates an example image of a surface of a mat covered by the epithelial cells, according to one or more implementations;

FIG. 8B illustrates an example image of a magnified view of the cell surface and Z-stack-derived reconstruction of side views of the mat shown in FIG. 8A;

FIG. 9 illustrates an example graph of transepithelial electrical resistance (TEER) measurements taken at key time points during culture compared to similar cell culture on a conventional flat porous membrane;

FIG. 10 illustrates a flow chart for an example 2-step electrospinning process according to one or more implementations;

FIG. 11A illustrates an example SEM image of PCL fibers generated from electrospinning PCL onto to the collector as configured by the process of FIG. 10, demonstrating a lower porosity layer according to one or more implementations;

FIG. 11B illustrates an example SEM image of PCL fibers generated from electrospinning PCL onto to the collector as configured by the process of FIG. 10, demonstrating a higher porosity layer according to one or more implementations; and

FIG. 12 illustrates a cross-sectional view of an example interface or mat for a target application according to the techniques described herein.

### DETAILED DESCRIPTION

Below are detailed descriptions of various concepts related to, and implementations of, techniques, approaches, methods, apparatuses, and systems that provide a system to study the intersection of two dimensional and three dimensional cell culture. The various concepts introduced above and discussed in greater detail below may be implemented in any of numerous ways, as the described concepts are not limited to any particular manner of implementation. Examples of specific implementations and applications are provided primarily for illustrative purposes.

In vitro tissue and organ-on-a-chip models are incredibly useful tools for medical discovery, research and development. Such models can be complex and reproduce cellular structures and conditions with high fidelity. Example models can be two-dimensional tissue models or three dimensional tissue models. However, organs can be composed of multiple different interacting cell types which serve different purposes and inhabit different biological environments. In particular, several critical organs of the body (for example airway, intestine, skin) include an epithelial cell sheet, which provides a barrier between self and the external milieu, and which is often the primary site of infection or other insult.

These cells are supported by an underlying layer of connective tissue inhabited by immune cells, neurons, fibroblasts and others that respond to insults and modulate epithelial biology. Direct contact among all of these cells results in coordinated responses to environmental changes, such as inflammation, immune activation, and neuronal signaling. Therefore, the in vitro models described herein that can recreate or model, for example, an infection, may include an epithelial monolayer culture in direct contact with an underlying environment incorporating relevant immune cells, fibroblasts or other cells. These techniques can utilize the integration of a flat surface to support the epithelial sheet with a three dimensional environment to house the responding cells.

Some approaches to the foregoing issues include placing epithelial cells onto a porous membrane and introducing additional cell types on the opposite side of the membrane in a two dimensional context, either on the membrane or on a separate surface. Another approach includes growing epithelial cells on the surface of a dense hydrogel, where additional cell type may be embedded within the hydrogel, but lack motility, perfusion, and cell addition/removal. However, such approaches do not integrate a surface appropriate for epithelial monolayer culture with a matrix designed to support three dimensional and motile cell culture.

The techniques described herein provide systems, devices, methods, and apparatuses that support the integration of epithelial cell layers on a two dimensional surface with a supporting three dimensional matrix that allows cell motility, introduction and removal of cells, and perfusion directly through the cell environment. The techniques described herein include utilizing a three dimensional cell culture environment, which can be formed directly onto or into an epithelial support layer without any intervening adhesive or gap. Electrospinning may be used to create a suitable three dimensional cell culture environment. Various implementations for integrating the epithelial layer with this environment are described herein.

In some implementations, a suitable epithelial support layer, such as a polycarbonate porous membrane, is placed over the collector of an electrospinner, and the three dimensional matrix is spun directly onto this surface. This can enable the pores of the electrospun matrix to communicate directly with the pores in the epithelial support layer. The electrospinning process creates a physical (e.g., van-der-Waals) type bonding between the membrane and matrix, removing the need for any adhesive which could potentially interfere with cell-cell communication.

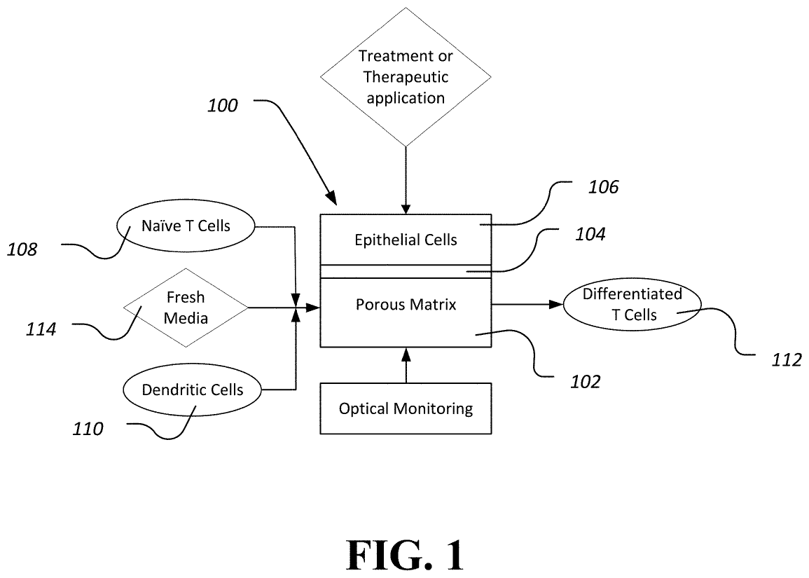

FIG. 1 illustrates a block diagram of an example interface 100 configured to receive cells, media, therapeutic and treatment applications, for example. The interface 100 may be a mat that includes a porous matrix 102, suitable for three-dimensional culturing of immune or other types of tissues cells. The porous matrix 102 may be interfaced with an epithelial support layer 104 configured to support epithelial cells 106. As illustrated by way of example in FIG. 1, naive T cells 108 and dendritic cells 110 may be introduced to the matrix 102, as well as fresh media 114. Moreover, cells may be removed from the matrix 102, e.g., differentiated T cells 112, as shown in FIG. 1.

The porous matrix 102 may enable introduction of fresh media 114, infusion and removal of cells over time, and opportunities for optical or chemical monitoring of the system. The close contact between the epithelial support layer 104 and the porous matrix 102 enables direct cell-cell interactions across this interface 100.

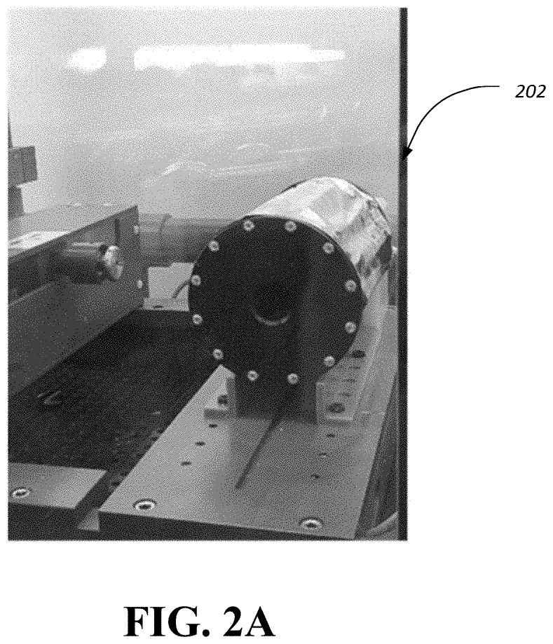

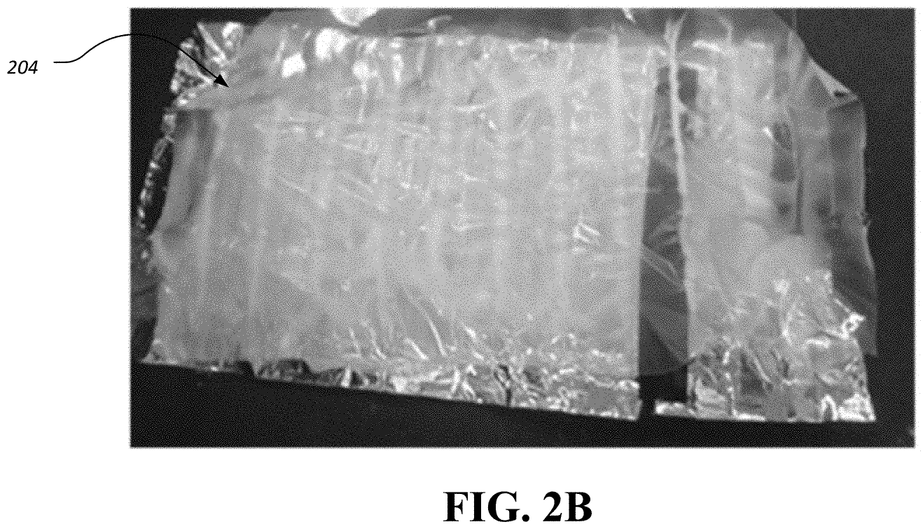

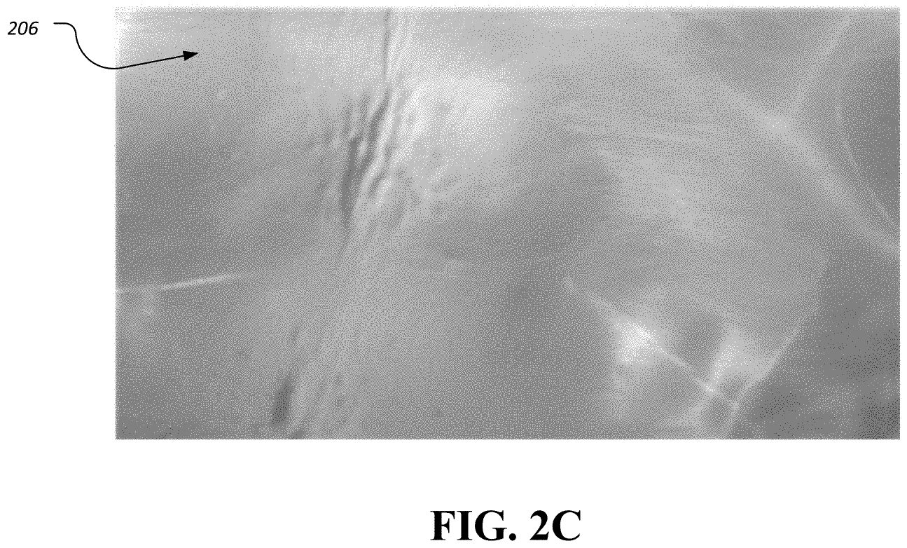

Referring to FIGS. 2A, 2B, and 2C, example devices and views for implementing a layering protocol are illustrated. FIG. 2A illustrates an example drum collector 202 in an electrospinning set-up. FIG. 2B illustrates an example of electrospun PCL fibers 204 onto a polycarbonate membrane, where the polycarbonate membrane had been mounted onto an aluminum foil layer. The polycarbonate membrane/aluminum foil layer is wrapped around the drum collector 202 (illustrated in FIG. 2A), and PCL fibers 204 are deposited onto the polycarbonate membrane during the electrospinning process.

FIG. 2B? is a non-magnified image of the electrospun PCL mat 206, where the variation of the material is visible. Material dispensed along the spoke region is visible on the left side of the image, whereas thinner layers of material are deposited in the valleys between the spokes.

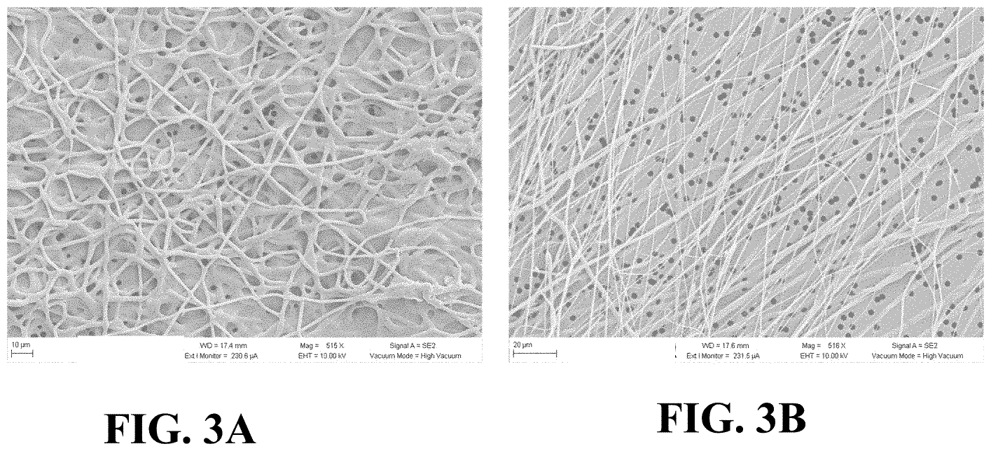

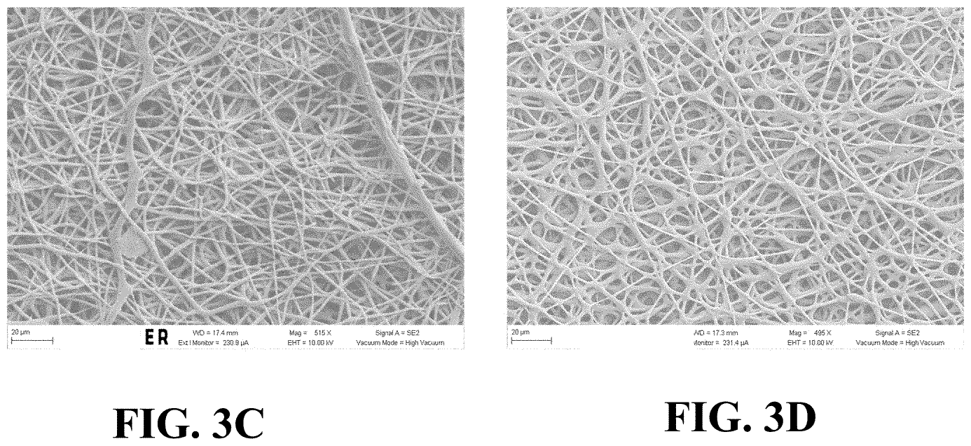

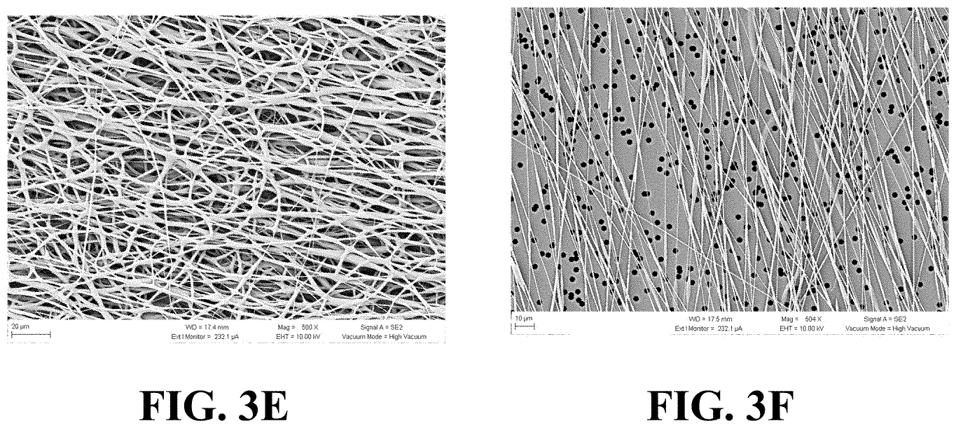

The series of images in FIGS. 2A-2C demonstrate the apparatus and layering of materials. This layout can be used to generate electrospun mats as depicted in FIGS. 3A-3D. FIGS. 3A and 3B illustrate examples of a thinner layer of material collected directly onto a porous material appropriate to support epithelial cells, such as track etched polycarbonate, while FIGS. 3C and 3D illustrate examples of a thicker layer of material. FIG. 3E illustrates an example of a thicker layer of material collected directly onto a porous material appropriate to support epithelial cells, such as track etched polycarbonate and FIG. 3F illustrates an example of a thinner layer of material collected directly onto a porous material appropriate to support epithelial cells, such as track etched polycarbonate.

During formation, a layer of foil (e.g., aluminum foil) may be placed on the collector to help evenly charge the wheel. Then, a polycarbonate membrane may be placed over the foil. When running the electrospinner, a 20 wt. % polycaprolactone (PCL) in a 75:25 dichloromethane (DCM):dimethylformamide (DMF) solution, in one example, may be pushed by a syringe through a positively charged nozzle at a rate of 2 mL/hr in a controlled humidity environment. This material may be attracted to the spinning collector with a negative charge and creates a Taylor Cone. This Taylor Cone may provide a single fiber stream that is deposited on the collector as the collector 202 spins. Many parameters can be changed to tune properties of the electrospun mats, including solution viscosity, solvent composition, applied voltage, flow rate, and collector rotation speed. Examples of electrospun mats generated with varying parameters are depicted in FIGS. 3A-3D.

For example, FIGS. 3A and 3B show examples of the dispersion of material between the collector spokes, while FIGS. 3C and 3D show examples of the dispersion of material overlaying the spoke regions of the collector. Parameters for FIGS. 3A and 3C can include, by way of an example: 20 wt % PCL in 75:25 DCM:DMF solution, Flow rate: 2 mL/hr, Voltage: 9.6 kV, Humidity: 22.3%, and Temperature: 25° C. Parameters for FIGS. 3B and 3D can include, by way of an example: 20 wt % PCL in 75:25 DCM:DMF solution, Flow rate: 2 mL/hr, Voltage: 10 kV, Humidity: 14.7%, and Temperature: 25° C. Parameters for FIGS. 3E and 3F can include, by way of an example: 20 wt % PCL in 75:25 DCM:DMF solution, Flow rate: 2 mL/hr, Voltage: 9 kV, Humidity: 18%, and Temperature: 19° C.

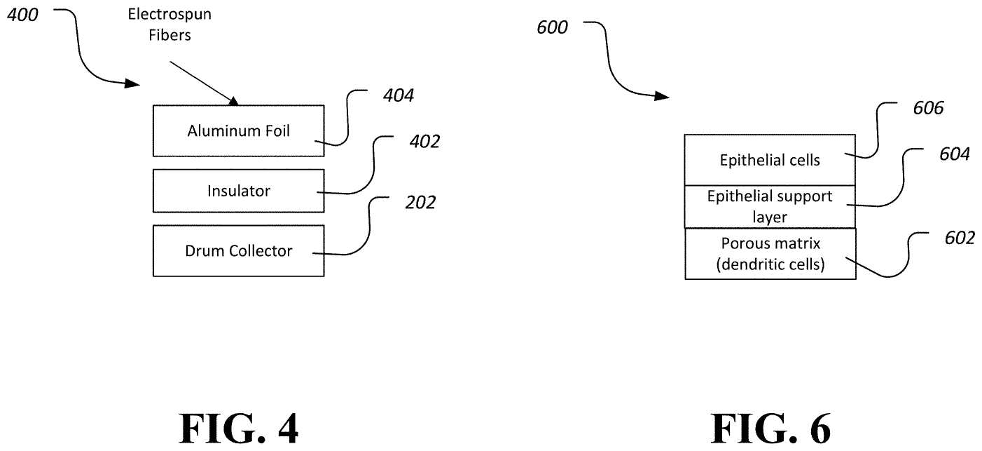

FIG. 4 illustrates a general stack-up arrangement 400 for an example configuration resulting in electrospun layers depositing at differing porosities, according to one or more implementations. In some implementations, adjustments can be made to generate electrospun mats of varying porosity within the same electrospin process run. In one example, an insulating layer 402 (e.g., polycarbonate, Teflon, etc.) can be placed between a conductive layer (e.g., aluminum foil 404). This stack up can be then wrapped around the drum collector 202, as shown by way of example in FIG. 4.

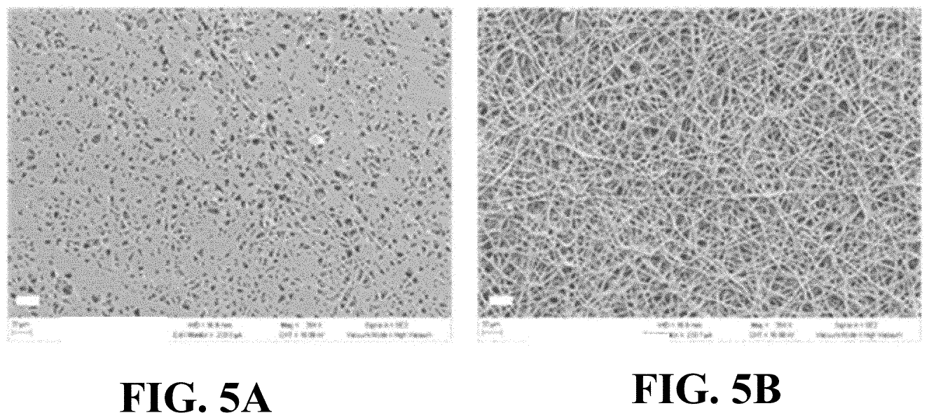

FIG. 5A illustrates an example scanning electron microscopy (SEM) image of PCL fibers generated from electrospinning PCL fibers onto to the collector as configured in FIG. 4, demonstrating a lower porosity layer, which is deposited first. FIG. 5B illustrates an example SEM image of PCL fibers generated from electrospinning PCL onto to the collector as configured in FIG. 4, demonstrating a higher porosity layer, which is deposited on top of the lower porosity layer.

From the configuration of FIG. 4, electrospun PCL fibers may be deposited onto the aluminum foil 404. The configuration results in an electrospun layer of lower porosity first depositing onto the foil 404, followed by a layer of higher porosity. The lower porosity layer may be suitable for epithelial cell culture, whereas dendritic cells can be seeded in the higher porosity layer, as shown in FIGS. 5A and 5B.

Referring back to FIG. 4, the drum collector 202 may be wrapped with the insulating layer 402, followed by the layer of aluminum foil 404. The electrospun fibers may then be deposited onto the outer layer of aluminum foil 404.

FIG. 6 illustrates an example stack-up arrangement 600 for an example configuration of the layers, including a porous matrix 602, with the epithelial support layer 604 thereon, followed by the epithelial cell layer 606 on the epithelial support layer 604. The porous matrix 602 may be similar to an example such as that shown in FIG. 5B, while the epithelial support layer 604 may be similar to an example such as that shown in FIG. 5A.

Again, FIG. 5A shows SEM images of PCL fibers generated from electrospinning PCL onto to the collector as configured in FIG. 4, where a lower porosity layer may be first deposited onto the foil 404, while a higher porosity layer may be deposited on top of the lower porosity layer, such as one illustrated in FIG. 5B. As explained above, the lower porosity layer may be used as an epithelial support layer 604, whereas the higher porosity layer may be suitable as a 3D tissue culture matrix (i.e., porous matrix 602) for dendritic cells and T-cells. The electrospin conditions for these example implementations can include, by way of example: 20 wt % PCL in 75:25 DCM:DMF solution, Flow rate: 2 mL/hr, Voltage: 10.3 kV, Humidity: 7.0%, and Temperature: 21.8° C. The scale bars are 20 μm.

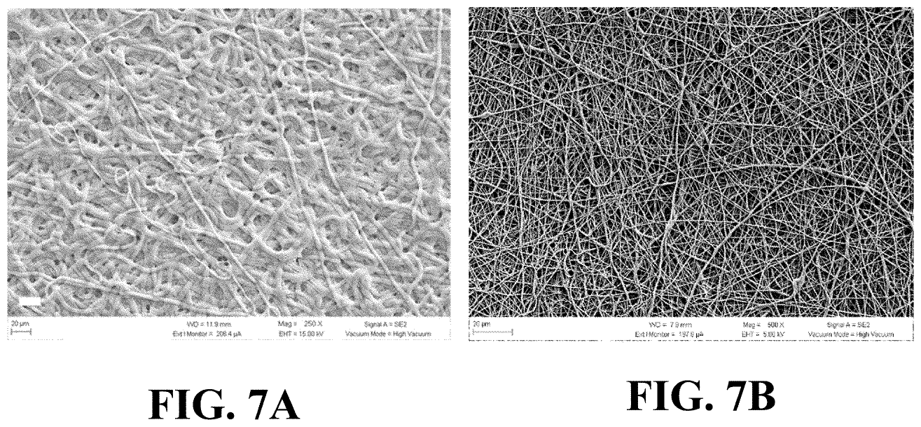

FIGS. 7A and 7B illustrate examples of electrospun PCL mats electrospun directly onto a foil-wrapped collector using different parameters. Various configurations may be achieved by tuning the electrospinning parameters such that a first layer of low porosity is deposited, then a second layer is deposited that has higher porosity. The reverse could also be executed (e.g., depositing first higher porosity, followed by depositing a layer of lower porosity, etc.). Example protocols include using a higher viscosity polymer solution to deposit fibers at lower porosity, followed by switching solutions (e.g., to a lower viscosity solution) to deposit a more porous network suitable for culturing the three dimensional tissue resident cells. Some example tissue resident cells may include dendritic cells and T-cells. The techniques described herein can be used to generate electrospun mats with varying porosity using separate protocols, as shown in FIGS. 7A and 7B. A two-step process can be used to combine the protocols into one electrospinning procedure.

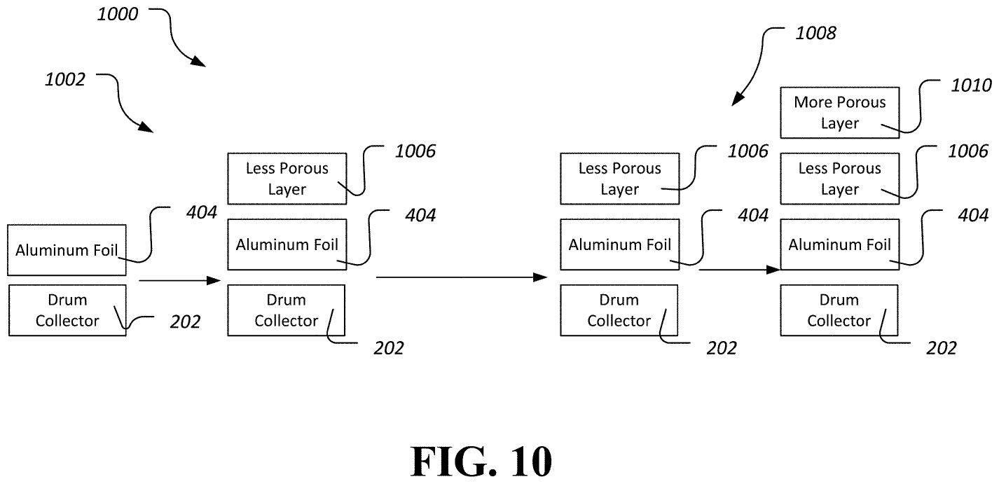

Specifically, a 2-step electrospinning process has been developed where a less porous polycaprolactone layer is first deposited by using a higher viscosity 20 wt. % polycaprolactone (PCL) in a 75:25 dichloromethane (DCM):N,N-dimethylformamide (DMF) solution (14 kV, 2 mL/hour flow rate, 60% humidity). After depositing a certain volume of the higher viscosity solution, a lower viscosity 12.5 wt. % polycaprolactone (PCL) in a 75:25 dichloromethane (DCM):N,N-dimethylformamide (DMF) solution (14 kV, 2 mL/hour flow rate, 60% humidity) is introduced on top of the already-deposited layer. An example 2-step process may be discussed in more detail with respect to FIG. 10.

Referring back to the examples illustrated in FIGS. 7A and B, the example mat shown in FIG. 7A was generated with the following electrospin conditions: 17.5 wt % PCL in 75:25 DCM:DMF solution, Flow rate: 3.5 mL/hr, and Voltage: 7.6 kV. The example mat shown in FIG. 7B was generated with the following electrospin conditions: 12.5 wt % PCL in 75:25 DCM:DMF solution, Flow rate: 2 mL/hr, Voltage: 8.2 kV, avg Humidity: 57%, avg and Temperature: 21.2° C. The scale bars are 20 μm.

An additional method to integrate a surface layer sufficient to support monolayer formation on an electrospun mat may result in the formation of a thin, cohesive hydrogel on the surface.

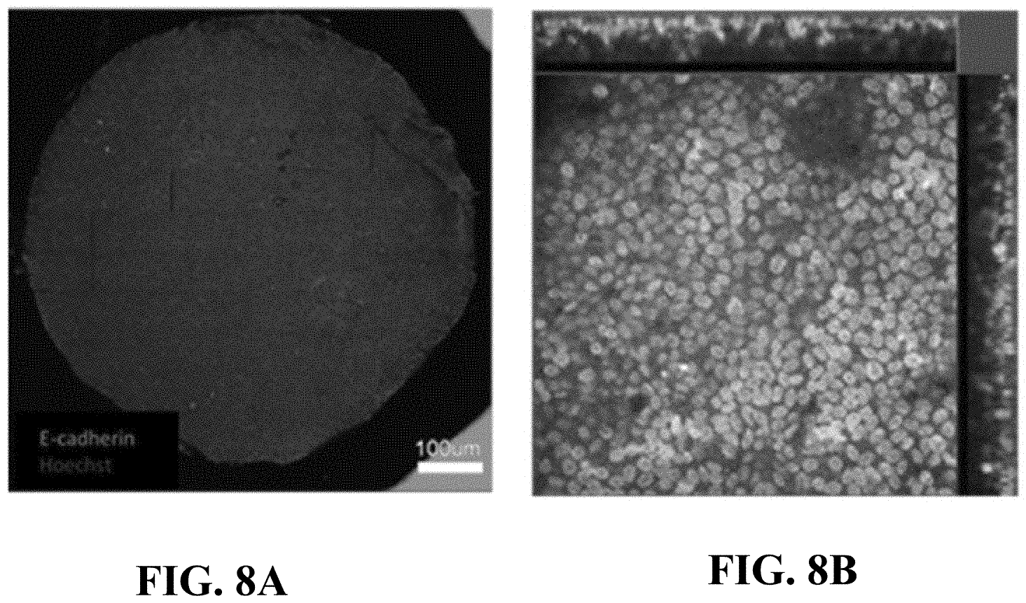

FIG. 8A illustrates an example image of a surface of a mat covered by the epithelial cells, according to one or more implementations. FIG. 8B illustrates an example image of a magnified view of the cell surface and Z-stack-derived reconstruction of side views of the mat shown in FIG. 8A.

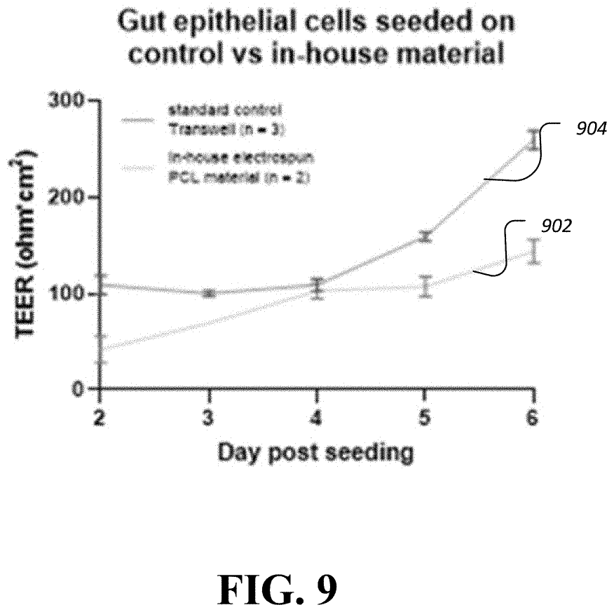

FIG. 9 shows an example graph of transepithelial electrical resistance (TEER) measurements taken at key time points during culture 902 compared to similar cell culture on a conventional flat porous membrane 904. The successful culture of an epithelial layer formed on the surface of an electrospun mat is depicted in FIGS. 8A and 8B. The cells grew into a tightly organized layer, demonstrated by organized E-cadherin expression, and importantly demonstrated measurable TEER, as shown in FIG. 8A.

In addition, the cell layer also included key cell types including M-cells, indicated by Spi-B and GP2 expression. Electrospinning may therefore produce a mat sufficient to support a relevant, differentiated and functional epithelial barrier. In addition, Dendritic cells may be cultured and matured in the electrospun mats described herein (not shown), using the techniques described herein. Therefore, the techniques described herein can provide an adequate environment for culture of non-epithelial cell types.

Referring again to FIGS. 8A and 8B, which illustrate images of intestinal epithelial cells grown on the surface of an electrospun polycarbonate mat, the PCL fibers may be electrospun onto foil with no insulating layer between the foil and the PCL. The sample mat may be made using a 12.5% wt PCL solution in a DCM:DMF solution in a higher humidity setting around room temperature. A 9.8 kV charge may be applied to the solution to create this mat. In an example, intestinal epithelial cells may be seeded onto the mat, and maintained in typical media conditions throughout 6 days of culture. At day 6, cells can be fixed and stained with key markers of cell type and organization.

FIG. 8A shows an image of a surface of an example mat covered by the epithelial cells. Formation of cell-cell junctions is consistent throughout the layer. FIG. 8B shows a magnified view of the cell surface and Z-stack-derived reconstruction of side views. Depth imaging indicates that some cells fall below the monolayer.

FIG. 9 illustrates a graph of example TEER measurements taken at key points during culture 902, and compared to similar cell culture on a conventional flat porous membrane 904, and the increasing TEER indicates that a functional barrier can be formed.

In practice, the epithelial support layer can be glued onto a suitable three dimensional cell culture matrix or clamped onto it. The epithelial support layer may also adhere to a suitable three dimensional cell culture matrix through van der Walls forces (i.e., no adhesive or clamping needed). The three dimensional cell culture matrix could be melted or dissolved to create a lower porosity surface for the epithelial layer. A hydrogel could be integrated or layered onto one surface to provide lower porosity surface appropriate for epithelial culture in one or more implementations. For instance, the inverted electrospun mat could be dipped into unformed hydrogel and then cured into a surface layer.

The techniques described herein can provide a single process material that can support direct interaction between epithelial layers and underlying cell types, without employing potentially toxic adhesives. Advantages include lack of toxic adhesives, direct cell contact between epithelial and underlying cells, the opportunity to introduce/remove underlying cells without disrupting the established epithelium, material that will allow underlying cells to move through the matrix, and the ability to perfuse the matrix directly.

Although the foregoing has been described in connection with polycaprolactone electrospun materials, it should be understood that the techniques described herein are suitable for other materials, including but not limited to collagen or gelatin, among others. The material may also be composed of at least two co-spun materials, or treated after spinning to coat the materials with a suitably biocompatible surface or interstitial material, such as matrigel. Co-spinning and post-treatment of the electrospun materials with a collagen or matrigel hydrogel material may include modification of the hydrogel stiffness as a function of depth away from the integrated epithelial support layer.

As explained, the porosity of the 3-dimensional matrix component can be altered by forming electrospun materials comprising polycaprolactone and a water-soluble polymer, such as polyvinylpyrrolidone (PVP). The water-soluble polymer acts a sacrificial component that can be removed via dissolving out with water, resulting in larger pores in the polycaprolactone matrix. Polycaprolactone and polyvinylpyrrolidone can be electrospun simultaneously to form interwoven matrices. An example electrospinning protocol for co-spun mats involves pushing a 20 wt. % polycaprolactone (PCL) in a 75:25 dichloromethane (DCM):N,N-dimethylformamide (DMF) solution through one syringe while simultaneously pushing a 10 wt. % polyvinylpyrrolidone (PVP) in ethanol solution through a separate syringe. The solutions are pushed through separated positively charged nozzles (with field of 14 kV) at a rate of 2 mL/hr in an environment maintained at 60% humidity environment. This material may be attracted to the spinning collector with a negative charge and creates a Taylor Cone. In this case, two single fiber streams are deposited on the collector as it spins. Many parameters or predetermined values can be changed to tune properties of the electrospun mats, including solution viscosity, solvent composition, applied voltage, flow rate, and collector rotation speed. After the co-spun matrix is formed, the polyvinylpyrrolidone portion can be removed by submerging the matrix in water. The matrix is dried in a manner to prevent collapse of the fibers. Drying methods may include freezing in liquid nitrogen followed by lyophilization, solvent-swapping to ethanol followed by desiccation or vacuum drying, or solvent-swapping to ethanol followed by critical point drying.

The matrices formed from co-spinning polycaprolactone with a water-soluble sacrificial polymer can be paired with a lower porosity layer. As one example, the 2-step process described herein may be translated to first generating the less porous polycaprolactone (PCL) layer, then depositing the co-spun polycaprolactone/polyvinylpyrrolidone layer as the second layer. The entire two-layer system may be subjected to the process for dissolving the sacrificial polymer followed by suitable drying conditions.

FIG. 10 illustrates an example process 1000 for a two-step electrospinning process according to one or more implementations. At a first stage 1002, the polycarbonate membrane/aluminum foil layer 402 is wrapped around the drum collector 202. In this stage, a first layer 1006 of PCL fibers are deposited onto the foil 402 using a higher viscosity solution, allowing the fibers to deposit at a lower porosity.

The process 1000 may then proceed to a second stage 1008 where a second layer 1010 of PCL fibers are deposited onto the first layer 1006. However, in this stage, a lower viscosity solution is used, allowing the fibers to deposit at a high porosity. By switching solutions (i.e., to a lower viscosity solution) to deposit a more porous network, the layers may be more suitable for culturing the three dimensional tissue resident cells. As explained throughout, some example tissue resident cells may include dendritic cells and T-cells.

As also explained, the two-step process 1000 may include first using the lower viscosity solution to create the first layer 1006 at a high porosity and then switching to a higher viscosity solution to deposit a less porous network for the second layer 1008 in other implementations.

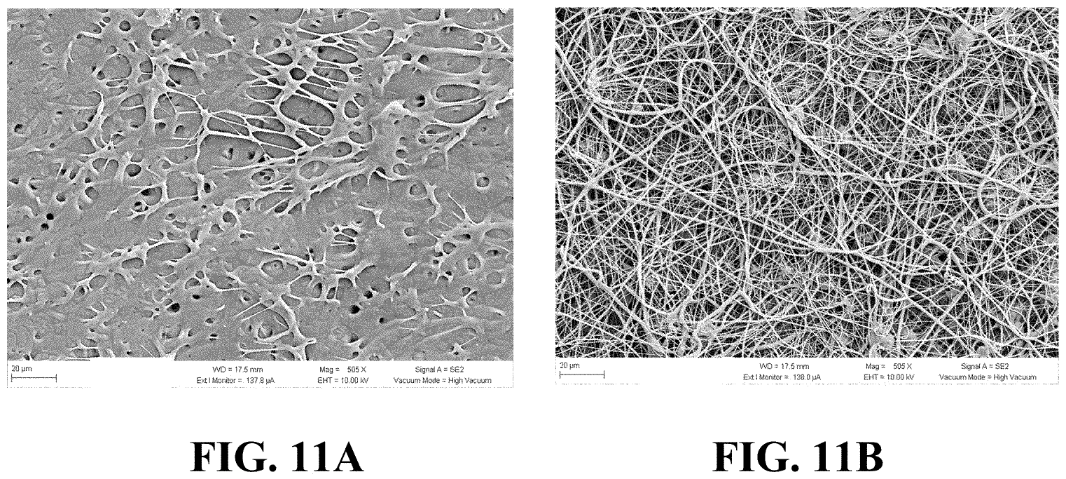

FIG. 11A illustrates an example SEM image of PCL fibers generated from electrospinning PCL onto to the collector as configured by the process of FIG. 10, demonstrating a lower porosity layer. FIG. 11B illustrates an example SEM image of PCL fibers generated from electrospinning PCL onto to the collector as configured by the process of FIG. 10, demonstrating a higher porosity layer. Referring back to FIG. 6, a similar stack-up arrangement 600 may be appreciated with the examples in FIGS. 11A and 11B, where the porous matrix 602 may be similar to an example such as that shown in FIG. 11B, while the epithelial support layer 604 may be similar to an example such as that shown in FIG. 11A. The 2-step electrospinning process (e.g., process 1000) may include depositing a less porous polycaprolactone layer using a higher viscosity 20 wt. % polycaprolactone (PCL) in a 75:25 dichloromethane (DCM):N,N-dimethylformamide (DMF) solution (14 kV, 2 mL/hour flow rate, 60% humidity). After depositing a certain volume of the higher viscosity solution, a lower viscosity 12.5 wt. % polycaprolactone (PCL) in a 75:25 dichloromethane (DCM):N,N-dimethylformamide (DMF) solution (14 kV, 2 mL/hour flow rate, 60% humidity) is introduced on top of the already-deposited layer.

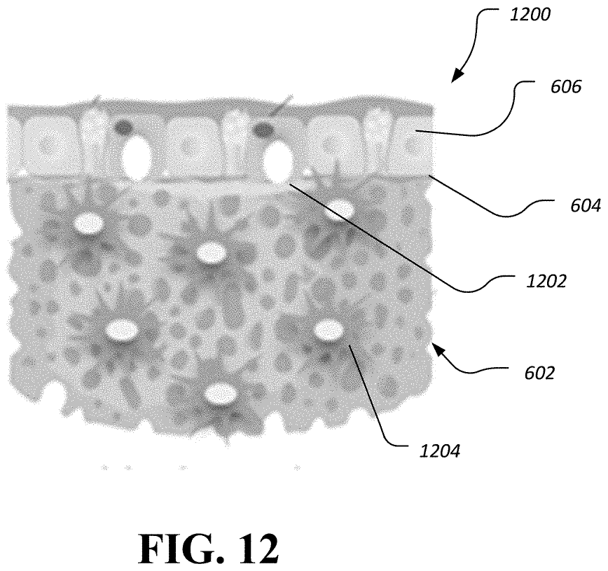

FIG. 12 illustrates a cross-sectional view of an example interface 1200 for a target application according to the techniques described herein. As illustrates in part with respect to FIG. 6, the interface 1200 includes including a porous matrix 602, with the epithelial support layer 604 thereon, and then the epithelial cell layer 606 on the epithelial support layer 604. The porous matrix 602 is the three dimensional cell culture matrix that supports cell mobility and profusion. A plurality of 3D matrix resident cells 1204 are illustrated therein. Further, an intimate cell-cell contact point 1202 is also illustrated between the epithelial cell layer 606 and the epithelial support layer 604. Thus, direct interaction between the epithelial layers and the underlying cell types can be achieved without employing potentially toxic adhesives. While operations are depicted in the drawings in a particular order, such operations are not required to be performed in the particular order shown or in sequential order, and all illustrated operations are not required to be performed. Actions described herein can be performed in a different order.

Further, while electrospinning is described herein for forming the mat, other mechanisms may be used to produce such mat and layers, including but not limited to 3D bioprinting, inverse opal colloidal templating, etc.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the terms “about” and “substantially” will be understood by persons of ordinary skill in the art and will vary to some extent depending upon the context in which they are used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation or embodiment, and references to “an implementation,” “some implementations,” “one implementation,” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Moreover, except where otherwise expressly indicated, all numerical quantities and ranges in this disclosure are to be understood as modified by the word “about.” Practice within the numerical limits stated is generally preferred. Also, unless expressly stated to the contrary, the description of a group or class of materials by suitable or preferred for a given purpose in connection with the disclosure implies that mixtures of any two or more members of the group or class may be equally suitable or preferred.

The term “substantially,” “generally,” or “about” may be used herein to describe disclosed or claimed embodiments. These terms may modify a value or relative characteristic disclosed or claimed in the present disclosure. In such instances, these terms may signify that the value or relative characteristic it modifies is within ±0%, 0.1%, 0.5%, 1%, 2%, 3%, 4%, 5% or 10% of the value or relative characteristic.

It should also be appreciated that integer values (and their modification via “about”) and integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

Where technical features in the drawings, detailed description, or any claim are followed by reference signs, the reference signs have been included to increase the intelligibility of the drawings, detailed description, and claims. Accordingly, neither the reference signs nor their absence has any limiting effect on the scope of any claim elements. The devices, systems, and methods described herein may be embodied in other specific forms without departing from the characteristics thereof. The foregoing implementations are illustrative rather than limiting of the described devices, systems, and methods.

What is claimed is:

1. A method for generating a cell support interface for use in a three dimensional cell culture environment, comprising:
electrospinning a mat having an epithelial support layer configured to create an intimate coupling between the epithelial cell and a porous matrix, including a first layer and a second layer, wherein the first layer is formed using a first solution at a first viscosity level and the second layer is formed using a second solution at a second viscosity level different from the first viscosity level.

2. The method of claim 1, wherein the first viscosity level is higher than the second viscosity level to form the first layer at a porosity lower than that of the second layer.

3. The method of claim 2, wherein a water-soluble polymer is formed with the second layer, and the method further comprises dissolving the water-soluble polymer in water.

4. The method of claim 3, further comprising drying the support layer.

5. The method of claim 1, wherein the first viscosity level is lower than the second viscosity level to form a first layer at a porosity higher than that of the second layer.

6. The method of claim 1, wherein the first layer is formed from electrospinning polycaprolactone.

7. The method of claim 1, wherein the second layer is formed from spinning polycaprolactone/polyvinylpyrrolidone.

8. The method of claim 1, further comprising treating the epithelial support layer with at least one of a collagen or matrigel hydrogel material to alter a hydrogel stiffness as a function of depth away from the epithelial support layer.

9. The method of claim 1, wherein the first and second solutions are applied via positively charged nozzles, each at a predetermined rate and humidity environment and are attracted to a negative charge generated during the electrospinning.

10. The method of claim 1, wherein the electrospinning includes electrospinning directly on an epithelial support layer comprising track-etched polycarbonate membranes.

11. A method for generating an electrospun mat for use in a three dimensional cell culture environment, comprising:
electrospinning a mat to facilitate intimate coupling between epithelial cell and a porous matrix, including co-spinning a first solution and a second solution, wherein the mat includes an epithelial support layer configured to intimately mate with epithelial cells cultured thereon.

12. The method of claim 11, further comprising integrating a hydrogel to the epithelial support layer to create a lower porosity surface for cell culture.

13. The method of claim 11, wherein the first and second solutions are applied via positively charged nozzles, each at a predetermined rate and humidity environment and are attracted to a negative charge generated during the electrospinning.

14. The method of claim 13, wherein a water-soluble polymer is formed with the porous matrix, and the method further comprises dissolving the water-soluble polymer in water.

15. A method for generating a cell support interface for use in a three dimensional cell culture environment, comprising:
electrospinning a first layer using a first solution at a first viscosity level; and
electrospinning a second layer using a second solution at a second viscosity level different from the first viscosity level, where the first layer and the second layer result in differing porosity levels for the first and second layers.

16. The method of claim 15, wherein the first viscosity level is higher than the second viscosity level to form the first layer at a porosity lower than that of the second layer.

17. The method of claim 15, wherein the first and second solutions are applied via positively charged nozzles, each at a predetermine rate and humidity environment and are attracted to a negative charge generated during the electrospinning.

18. The method of claim 15, where a water-soluble polymer is formed with the second layer and further comprising dissolving the water-soluble polymer.

19. The method of claim 15, wherein the first layer is formed from electrospinning polycaprolactone and wherein the second layer is formed from spinning polycaprolactone/polyvinylpyrrolidone.

20. The method of claim 15, further comprising treating the epithelial support layer with at least one of a collagen or matrigel hydrogel material to alter a hydrogel stiffness as a function of depth away from the epithelial support layer.
